SEQUENCE LISTING (15 sequences)

SEQ ID NO: 1 - DNA, 1464 bp, Arabidopsis thaliana
atgaagattacaaaaccacatgtggccatgttcgctagccccggaatgggccacatcatcccggtgatcgagctcggaaaacgcttagctggttcccacggcttcgatgtcaccattttcgtccttgaaaccgacgcagcctcagctcaatctcaattccttaactcaccaggctgcgacgcggcccttgttgatatcgttggcctcccaacgcccgatatctccggtttagtcgacccatcagccttttttgggatcaagctcttggtcatgatgcgtgagaccattcctaccatccggtcaaagatagaggagatgcaacacaaaccaacggctctgatcgtagacttgtttggtttggacgcgataccgctcggtggtgagttcaacatgttgacttatatcttcatcgcttcaaacgcacgttttctcgcggtggctttgtttttcccaacgttggacaaagacatggaagaagagcacataatcaagaagcaacctatggttatgcctggatgtgaaccggttcggtttgaagatacacttgaaacattccttgacccaaacagccaactctaccgggaatttgttcctttcggttcggttttcccaacgtgtgatggtattattgtgaatacatgggatgatatggagcccaaaactttgaaatctcttcaagacccaaagctcttgggtcgaattgctggtgtaccggtttatccaattggtcctttgtctagaccggttgatccatctaaaactaatcatccggttttggattggttaaacaaacagccggacgagtcggtactttacatttcatttggaagcggtggctctctctcggctaaacaactaaccgaattggcttggggacttgagatgagtcagcaacggttcgtttgggtggttcgacccccggtggacggttcagcttgcagtgcatatttatccgctaacagtggtaaaatacgagacggtacacctgattatctcccggaaggttttgttagccggactcatgagagaggctttatggtctcttcttgggctccccaagcggagatcttggcccaccaagccgtaggtgggtttctaactcactgcggttggaattcgattctcgagagcgtcgttggtggcgttccgatgatcgcgtggccactttttgcggagcagatgatgaacgcgacactcctcaacgaagagcttggcgttgccgtccgctctaagaaactaccgtcggagggagtgattacgagggcggagatcgaggcgttggtgagaaagatcatggtggaggaggaaggtgctgagatgagaaagaagataaagaagctgaaagagaccgctgccgaatcgctgagttgcgacggtggagtggcgcatgaatcgttgtcaagaatcgccgacgagagcgagcatcttttggagcgtgtcaggtgcatggcacgtggtgcctag

SEQ ID NO: 2 - protein, 478 aa, Arabidopsis thaliana
MFASPGMGHIIPVIELGKRLAGSHGFDVTIFVLETDAASAQSQFLNSPGCDAALVDIVGLPTPDISGLVDPSAFFGIKLLVMMRETIPTIRSKIEEMQHKPTALIVDLFGLDAIPLGGEFNMLTYIFIASNARFLAVALFFPTLDKDMEEEHIIKKQPMVMPGCEPVRFEDTLETFLDPNSQLYREFVPFGSVFPTCDGIIVNTWDDMEPKTLKSLQDPKLLGRIAGVPVYPIGPLSRPVDPSKTNHPVLDWLNKQPDESVLYISFGSGGSLSAKQLTELAWGLEMSQQRFVWVVRPPVDGSACSAYLSANSGKIRDGTPDYLPEGFVSRTHERGFMVSSWAPQAEILAHQAVGGFLTHCGWNSILESVVGGVPMIAWPLFAEQMMNATLLNEELGVAVRSKKLPSEGVITRAEIEALVRKIMVEEEGAEMRKKIKKLKETAAESLSCDGGVAHESLSRIADESEHLLERVRCMARGA

SEQ ID NO: 3 - DNA, 1446 bp, Arabidopsis thaliana
atgcatatcacaaaaccacacgccgccatgttttccagtcccggaatgggccatgtcatcccggtgatcgagcttggaaagcgtctctccgctaacaacggcttccacgtcaccgtcttcgtcctcgaaaccgacgcagcctccgctcaatccaagttcctaaactcaaccggcgtcgacatcgtcaaacttccatcgccggacatttatggtttagtggaccccgacgaccatgtagtgaccaagatcggagtcattatgcgtgcagcagttccagccctccgatccaagatcgctgccatgcatcaaaagccaacggctctgatcgttgacttgtttggcacagatgcgttatgtctcgcaaaggaatttaacatgttgagttatgtgtttatccctaccaacgcacgttttctcggagtttcgatttattatccaaatttggacaaagatatcaaggaagagcacacagtgcaaagaaacccactcgctataccggggtgtgaaccggttaggttcgaagatactctggatgcatatctggttcccgacgaaccggtgtaccgggattttgttcgtcatggtctggcttacccaaaagccgatggaattttggtaaatacatgggaagagatggagcccaaatcattgaagtcccttctaaacccaaagctcttgggccgggttgctcgtgtaccggtctatccaatcggtcccttatgcagaccgatacaatcatccgaaaccgatcacccggttttggattggttaaacgaacaaccgaacgagtcggttctctatatctccttcgggagtggtggttgtctatcggcgaaacagttaactgaattggcgtggggactcgagcagagccagcaacggttcgtatgggtggttcgaccaccggtcgacggttcgtgttgtagcgagtatgtctcggctaacggtggtggaaccgaagacaacacgccagagtatctaccggaagggttcgtgagtcgtactagtgatagaggtttcgtggtcccctcatgggccccacaagctgaaatcctgtcccatcgggccgttggtgggtttttgacccattgcggttggagctcgacgttggaaagcgtcgttggcggcgttccgatgatcgcatggccactttttgccgagcagaatatgaatgcggcgttgctcagcgacgaactgggaatcgcagtcagattggatgatccaaaggaggatatttctaggtggaagattgaggcgttggtgaggaaggttatgactgagaaggaaggtgaagcgatgagaaggaaagtgaagaagttgagagactcggcggagatgtcactgagcattgacggtggtggtttggcgcacgagtcgctttgcagagtcaccaaggagtgtcaacggtttttggaacgtgtcgtggacttgtcacgtggtgcttag

SEQ ID NO: 4 - protein, 481 aa, Arabidopsis thaliana
MHITKPHAAMFSSPGMGHVIPVIELGKRLSANNGFHVTVFVLETDAASAQSKFLNSTGVDIVKLPSPDIYGLVDPDDHVVTKIGVIMRAAVPALRSKIAAMHQKPTALIVDLFGTDALCLAKEFNMLSYVFIPTNARFLGVSIYYPNLDKDIKEEHTVQRNPLAIPGCEPVRFEDTLDAYLVPDEPVYRDFVRHGLAYPKADGILVNTWEEMEPKSLKSLLNPKLLGRVARVPVYPIGPLCRPIQSSETDHPVLDWLNEQPNESVLYISFGSGGCLSAKQLTELAWGLEQSQQRFVWVVRPPVDGSCCSEYVSANGGGTEDNTPEYLPEGFVSRTSDRGFVVPSWAPQAEILSHRAVGGFLTHCGWSSTLESVVGGVPMIAWPLFAEQNMNAALLSDELGIAVRLDDPKEDISRWKIEALVRKVMTEKEGEAMRRKVKKLRDSAEMSLSIDGGGLAHESLCRVTKECQRFLERVVDLSRGA

SEQ ID NO: 5 - DNA, 1446 bp, Arabidopsis thaliana
atgcatatcacaaaaccacacgccgccatgttttccagtcccggaatgggccatgtcctcccggtgatcgagctagctaagcgtctctccgctaaccacggcttccacgtcaccgtcttcgtccttgaaactgacgcagcctccgttcagtccaagctccttaactcaaccggtgttgacatcgtcaaccttccatcgcccgacatttctggcttggtagaccccaacgcccatgtggtgaccaagatcggagtcattatgcgtgaagctgttccaaccctccgatccaagatcgttgccatgcatcaaaacccaacggctctgatcattgacttgtttggcacagatgcgttatgtcttgcagcggagttaaacatgttgacttatgtctttatcgcttccaacgcgcgttatctcggagtttcgatatattatccaactttggacgaagttatcaaagaagagcacacagtgcaacgaaaaccgctcactataccggggtgtgaaccggttagatttgaagatattatggatgcatatctggttccggacgaaccggtgtaccacgatttggttcgtcactgtctggcctacccaaaagcggatggaatcttggtgaatacatgggaagagatggagcccaaatcattaaagtcccttcaagacccgaaacttttgggccgggtcgctcgtgtaccggtttatccggttggtccgttatgcagaccgatacaatcatccacgaccgatcacccggtttttgattggttaaacaaacaaccaaacgagtcggttctctacatttccttcgggagtggtggttctctaacggctcaacagttaaccgaattggcgtgggggctcgaggagagccagcaacggtttatatgggtggttcgaccgcccgttgacggctcgtcttgcagtgattatttctcggctaaaggcggtgtaaccaaagacaacacgccagagtatctaccagaagggttcgtgactcgtacttgcgatagaggtttcatgatcccatcatgggcaccgcaagctgaaatcctagcccatcaggccgttggtgggtttttaacacattgtggttggagctcgacgttggaaagcgtcctttgcggcgttccaatgatagcgtggccgcttttcgccgagcagaatatgaacgcggcgttgcttagcgatgaactgggaatctctgttagagtggatgatccaaaggaggcgatttctaggtcgaagattgaggcgatggtgaggaaggttatggctgaggacgaaggtgaagagatgagaaggaaagtgaagaagttgagagacacggcggagatgtcacttagtattcacggtggtggttcggcgcatgagtcgctttgcagagtcacgaaggagtgtcaacggtttttggaatgtgtcggggacttgggacgtggtgcttag

SEQ ID NO: 6 - protein, 481 aa, Arabidopsis thaliana
MHITKPHAAMFSSPGMGHVLPVIELAKRLSANHGFHVTVFVLETDAASVQSKLLNSTGVDIVNLPSPDISGLVDPNAHVVTKIGVIMREAVPTLRSKIVAMHQNPTALIIDLFGTDALCLAAELNMLTYVFIASNARYLGVSIYYPTLDEVIKEEHTVQRKPLTIPGCEPVRFEDIMDAYLVPDEPVYHDLVRHCLAYPKADGILVNTWEEMEPKSLKSLQDPKLLGRVARVPVYPVGPLCRPIQSSTTDHPVFDWLNKQPNESVLYISFGSGGSLTAQQLTELAWGLEESQQRFIWVVRPPVDGSSCSDYFSAKGGVTKDNTPEYLPEGFVTRTCDRGFMIPSWAPQAEILAHQAVGGFLTHCGWSSTLESVLCGVPMIAWPLFAEQNMNAALLSDELGISVRVDDPKEAISRSKIEAMVRKVMAEDEGEEMRRKVKKLRDTAEMSLSIHGGGSAHESLCRVTKECQRFLECVGDLGRGA

SEQ ID NO: 7 - DNA, 28 bp, Arabidopsis thaliana
ctcgagcccgggatgaagattacaaaac

SEQ ID NO: 8 - DNA, 29 bp, Arabidopsis thaliana
atcttgtcaccacaaaggctgatgggtcg

SEQ ID NO: 9 - DNA, 30 bp, Arabidopsis thaliana
cgacccatcagcctttgtggtgaccaagat

SEQ ID NO: 10 - DNA, 30 bp, Arabidopsis thaliana
ggtatagcgagtgggtttcgttgcactgtg

SEQ ID NO: 11 - DNA, 30 bp, Arabidopsis thaliana
cacagtgcaacgaaacccactcgctatacc

SEQ ID NO: 12 - DNA, 32 bp, Arabidopsis thaliana
atttaaattctagagatgattgtatcggtctg

SEQ ID NO: 13 - DNA, 248 bp, Arabidopsis thaliana
atgaagattacaaaaccacatgtggccatgttcgctagccccggaatgggccacatcatcccggtgatcgagctcggaaaacgcttagctggttcccacggcttcgatgtcaccattttcgtccttgaaaccgacgcagcctcagctcaatctcaattccttaactcaccaggctgcgacgcggcccttgttgatatcgttggcctcccaacgcccgatatctccggtttagtcgacccatcagcctt

SEQ ID NO: 14 - DNA, 234 bp, Arabidopsis thaliana
tgtggtgaccaagatcggagtcattatgcgtgaagctgttccaaccctccgatccaagatcgttgccatgcatcaaaacccaacggctctgatcattgacttgtttggcacagatgcgttatgtcttgcagcggagttaaacatgttgacttatgtctttatcgcttccaacgcgcgttatctcggagtttcgatatattatccaactttggacgaagttatcaaagaagagca

SEQ ID NO: 15 - DNA, 247 bp, Arabidopsis thaliana
cacagtgcaaagaaacccactcgctataccggggtgtgaaccggttaggttcgaagatactctggatgcatatctggttcccgacgaaccggtgtaccgggattttgttcgtcatggtctggcttacccaaaagccgatggaattttggtaaatacatgggaagagatggagcccaaatcattgaagtcccttctaaacccaaagctcttgggccgggttgctcgtgtaccggtctatccaatcggt

(19) United States
(12) Patent Application Publication
(10) Pub. No.: US 2010/0181035 A1
(43) Pub. Date: Jul. 22, 2010

(54) GLUCOSYLTRANSFERASES

(75) Inventors: Dianna BOWLES, York (GB); Rosamond JACKSON, York (GB); Eng Kiat LIM, York (GB); Fabian Vaistij, York (GB)

Correspondence Address: SPECKMAN LAW GROUP PLLC, 1201 THIRD AVENUE, SUITE 330, SEATTLE, WA, 98101, US

(73) Assignee: UNIVERSITY OF YORK, York (GB)

(21) Appl. No.: 12/638,069

(22) Filed: Dec. 15, 2009

Related U.S. Application Data
(63) Continuation of application No. 10/575,349, filed on Nov. 6, 2006, now abandoned.

(30) Foreign Application Priority Data
Oct. 13, 2003 (GB) 0323813.6

(51) Int. Cl. D21C 3/00; C12N 5/10; A01H 5/00; C12Q 1/02

(52) U.S. Cl. 162/100; 435/419; 800/298; 800/319; 435/29

(57) ABSTRACT

We describe transgenic cells which are transformed with nucleic acid molecules which encode glucosyltransferase polypeptides involved in phenylpropanoid biosynthesis.

### REFERENCE TO RELATED APPLICATIONS

This application is a continuation of application Ser. No. 10/575,349, filed Nov. 6, 2006, which is the US National Stage of International Application no. PCT/GB2004/004330, filed Oct. 12, 2004, which claims priority to Great Britain patent application no. 0323813.6, filed Oct. 13, 2003.

### FIELD OF THE INVENTION

The invention relates to transgenic cells which have been transformed with nucleic acid molecules which encode glucosyltransferases (GTase) which glycosylate monolignols which are intermediates in lignin biosynthesis.

### BACKGROUND

GTases are enzymes which post-translationally transfer glucosyl residues from an activated nucleotide sugar to monomeric and polymeric acceptor molecules such as other sugars, proteins, lipids and other organic substrates. These glucosylated molecules take part in diverse metabolic pathways and processes. The transfer of a glucosyl moiety can alter the acceptor's bioactivity, solubility and transport properties within the cell and throughout the plant. One family of GTases in higher plants is defined by the presence of a C-terminal consensus sequence. The GTases of this family function in the cytosol of plant cells and catalyse the transfer of glucose to small molecular weight substrates, such as phenylpropanoid derivatives, coumarins, flavonoids, other secondary metabolites and molecules known to act as plant hormones.

Wood used in the paper industry is initially particulated, typically by chipping, before conversion to a pulp which can be utilised to produce paper. The pulping process involves the removal of lignin. Lignin is a major non-carbohydrate component of wood and comprises approximately one quarter of the raw material in wood pulp. The removal of lignin is desirable since the quality of the paper produced from the pulp is largely determined by the lignin content. Many methods have been developed to efficiently and cost effectively remove lignin from wood pulp. These methods can be chemical, mechanical or biological. For example, chemical methods to pulp wood are disclosed in WO9811294, EP0957198 and WO0047812. Although chemical methods are efficient means to remove lignin from pulp it is known that chemical treatments can result in degradation of polysaccharides and is expensive. Moreover, to remove residual lignin from pulp it is necessary to use strong bleaching agents which require removal before the pulp can be converted into paper. These agents are also damaging to the environment.

Biological methods to remove lignin are known, but have inherent disadvantages. For example it is important to provide micro-organisms (e.g. bacteria and/or fungi) which only secrete ligninolytic enzymes which do not affect cellulose fibres. This method is also very time consuming (can take 3-4 weeks) and expensive due to the need to provide bioreactors. Biological treatment can also include pre-treatment of wood chips to make them more susceptible to further biological or chemical pulping.

In some applications, for example the construction industry or in furniture making, it may be desirable to increase the lignin content of a plant cell to increase the mechanical strength of wood.

Both lignin content and the level of cross-linking of polysaccharide polymers within plant cell walls, also play an important role in determining texture and quality of raw materials through altering the cell walls and tissue mechanical properties. For example, there is considerable interest in reducing cell separation in edible tissues since this would prevent over-softening and loss of juiciness. Phenolics, such as ferulic acid, play an important role in cell adhesion since they can be esterified to cell wall polysaccharides during synthesis and oxidatively cross-linked in the wall, thereby increasing rigidity. Most non-lignified tissues contain these phenolic components and their levels can be modified by altering flux through the same metabolic pathways as those culminating in lignin. Therefore, in the same way as for the manipulation of lignin composition and content, GTase nucleic acid in sense and/or antisense configurations can be used to affect levels of ferulic acid and related phenylpropanoid derivatives that function in oxidative cross-linking. These changes in content have utility in the control of raw material quality of edible plant tissues.

Lignin and oxidative cross-linking in plant cell walls also play important roles in stress and defense responses of most plant species. For example, when non-woody tissues are challenged by pests or pathogen attack, or suffer abiotic stress such as through mechanical damage or UV radiation, the plant responds by localised and systemic alteration in cell wall and cytosolic properties, including changes in lignin content and composition and changes in cross-linking of other wall components. Therefore, it can also be anticipated that cell- or tissue-specific changes in these responses brought about by changed levels of the relevant GTase activities will have utility in protecting the plant to biotic attack and biotic/abiotic stresses.

GTases also have utility with respect to the modification of antioxidants. Reactive oxygen species are produced in all aerobic organisms during respiration and normally exist in a cell in balance with biochemical anti-oxidants. Environmental challenges, such as by pollutants, oxidants, toxicants, heavy metals and so on, can lead to excess reactive oxygen species which perturb the cellular redox balance, potentially leading to wide-ranging pathological conditions. In animals and humans, cardiovascular diseases, cancers, inflammatory and degenerative disorders are linked to events arising from oxidative damage.

Because of the current prevalence of these diseases, there is considerable interest in anti-oxidants, consumed in the diet or applied topically such as in UV-screens. Anti-oxidant micronutrients obtained from vegetables and fruits, teas, herbs and medicinal plants are thought to provide significant protection against health problems arising from oxidative stress. Well known anti-oxidants from plant tissues include for example: quercetin, luteolin, and the catechin, epicatechin and cyanidin groups of compounds.

Certain plant species, organs and tissues are known to have relatively high levels of one or more compounds with anti-oxidant activity. Greater accumulation of these compounds in those species, their wider distribution in crop plants and plant parts already used for food and drink production, and the increased bioavailability of anti-oxidants (absorption, metabolic conversions and excretion rate) are three features considered to be highly desirable.

The identity of a number of glucosyltransferase genes involved in lignin biosynthesis within Arabidopsis have been described in Lim et al. 2001. The isolation and characterisation of two of these genes, 72E2 and 72E3, both members of a small subfamily within Group E of the phylogenetic tree of Arabidopsis UGTs, were further disclosed in WO01/59140. The UGTs encoded by these genes glycosylate the metabolites of the phenylpropanoid pathway by the transfer of glucose from UDP-glucose to a hydroxyl group on the metabolite. This leads either to the formation of a glucose ester. The identification and characterisation of 72E2 and 72E3 led to the ability, via regulating gene expression, to be able to modulate the extent of lignification within a plant, thereby altering the mechanical properties, responsiveness to wounding and pathogen stress and xenobiotic de-toxification ability of the plant. We disclose a further sequence involved in glycosylation of metabolites in the phenylpropanoid pathway which alone or in combination with 72E2 and 72E3 modulate lignin biosynthesis.

### SUMMARY

According to an aspect of the invention there is provided a transgenic cell wherein the genome of said cell comprises a nucleic acid molecule wherein said nucleic acid molecule is selected from the group consisting of;
i) a nucleic acid molecule comprising a nucleic acid sequence as represented in FIG. 1 (SEQ ID NO: 1);
ii) a nucleic acid molecule comprising a nucleic acid sequence which hybridises to the sequence in (i) above and which glucosylates at least one monolignol;
iii) a nucleic acid molecule comprising a nucleic acid sequences which are degenerate as a result of the genetic code to the sequences defined in (i) and (ii) above.

In a preferred embodiment of the invention said aldehyde of a monolignol is selected from the group consisting of; p-coumaryl aldehyde, coniferyl aldehyde and sinapyl aldehyde.

In an alternative preferred embodiment of the invention said monolignol is coniferyl alcohol.

Preferably said hybridisation is stringent hybridisation. Stringent hybridisation/washing conditions are well known in the art. For example, nucleic acid hybrids that are stable after washing in 0.1×SSC, 0.1% SDS at 60° C. It is well known in the art that optimal hybridisation conditions can be calculated if the sequence of the nucleic acid is known. For example, hybridisation conditions can be determined by the GC content of the nucleic acid subject to hybridisation.

In a further preferred embodiment of the invention said nucleic acid is cDNA.

In a yet further preferred embodiment of the invention said nucleic acid is genomic DNA.

In a preferred embodiment of the invention said nucleic acid molecule comprises a nucleic acid sequence as shown in FIG. 1 (SEQ ID NO: 1). Preferably said nucleic acid molecule consists of a nucleic acid sequence as shown in FIG. 1 (SEQ ID NO: 1).

In a further preferred embodiment of the invention said nucleic acid molecule is over expressed.

In a preferred embodiment of the invention said over-expression is at least 2-fold higher when compared to a non-transformed reference cell of the same species. Preferably said over-expression is: at least 3-fold, 4-fold, 5-fold, 6-fold, 7-fold, 8-fold, 9-fold, or at least 10-fold when compared to a non-transformed reference cell of the same species.

In a further preferred embodiment of the invention said cell over-expresses a nucleic acid molecule selected from the group consisting of:
i) a nucleic acid molecule comprising a nucleic acid sequence as represented in FIG. 1 (SEQ ID NO: 1) and FIG. 3 (SEQ ID NO: 3) and/or FIG. 5 (SEQ ID NO: 5);
ii) a nucleic acid molecule comprising a nucleic acid sequence which hybridises to the sequence in (i) above and which glucosylates at least one monolignol;
iii) a nucleic acid molecule comprising a nucleic acid sequences which are degenerate as a result of the genetic code to the sequences defined in (i) and (ii) above.

In an alternative preferred embodiment of the invention said cell over-expresses a nucleic acid molecule as represented by the nucleic acid sequence shown in FIG. 3 (SEQ ID NO: 3) and FIG. 5 (SEQ ID NO: 5), or a nucleic acid molecule which hybridises to a nucleic acid molecule as represented by the nucleic acid sequence in FIG. 3 (SEQ ID NO: 3) and FIG. 5 (SEQ ID NO: 5).

This over expression may be as a result of an increased copy number of said nucleic acid molecule. Alternatively said nucleic acid sequence may be operably linked to a heterologous promoter.

A vector comprising the nucleic acid molecule operably linked to a heterologous promoter would be used to transfect/transform a selected cell.

“Vector” includes, inter alia, any plasmid, cosmid, phage or Agrobacterium binary vector in double or single stranded linear or circular form which may or may not be self-transmissable or mobilizable, and which can transform a prokaryotic or eukaryotic host either by integration into the cellular genome or exist extrachromosomally (e.g. autonomous replicating plasmid with an origin of replication, i.e. an episomal vector).

Suitable vectors can be constructed, containing appropriate regulatory sequences, including promoter sequences, terminator fragments, polyadenylation sequences, enhancer sequences, marker genes and other sequences as appropriate. For further details see, for example, Molecular Cloning: Laboratory Manual: 2^{nd} edition, Sambrook et al. 1989, Cold Spring Harbor Laboratory Press or Current Protocols in Molecular Biology, Second Edition, Ausubel et al. Eds., John Wiley & Sons, 1992.

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

A vector including nucleic acid according to the invention need not include a promoter or other regulatory sequence, particularly if the vector is to be used to introduce the nucleic acid into cells for recombination into the gene.

Preferably the nucleic acid in the vector is under the control of, and operably linked to, an appropriate promoter or other regulatory elements for transcription in a host cell such as a microbial, (e.g. bacterial), or plant cell. The vector may be a bi-functional expression vector which functions in multiple hosts. In the case of GTase genomic DNA this may contain its own promoter or other regulatory elements and in the case of cDNA this may be under the control of an appropriate promoter or other regulatory elements for expression in the host cell.

By “promoter” is meant a nucleotide sequence upstream from the transcriptional initiation site and which contains all the regulatory regions required for transcription. Suitable promoters include constitutive, tissue-specific, inducible, developmental or other promoters for expression in plant cells comprised in plants depending on design. Such promoters include viral, fungal, bacterial, animal and plant-derived promoters capable of functioning in plant cells.

Constitutive promoters include, for example CaMV 35S promoter (Odell et al. (1985) Nature 313, 9810-812); rice actin (McElroy et al. (1990) Plant Cell 2: 163-171); ubiquitin (Christian et al. (1989) Plant Mol. Biol. 18 (675-689); pEMU (Last et al. (1991) Theor Appl. Genet. 81: 581-588); MAS (Velten et al. (1984) EMBO J. 3. 2723-2730); ALS promoter (U.S. application Ser. No. 08/409,297), and the like. Other constitutive promoters include those in U.S. Pat. Nos. 5,608,149; 5,608,144; 5,604,121; 5,569,597; 5,466,785; 5,399,680, 5,268,463; and 5,608,142.

Chemical-regulated promoters can be used to modulate the expression of a gene in a plant through the application of an exogenous chemical regulator. Depending upon the objective, the promoter may be a chemical-inducible promoter, where application of the chemical induced gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88: 10421-10425 and McNellis et al. (1998) Plant J. 14(2): 247-257) and tetracycline-inducible and tetracycline-repressible promoters (see, for example, Gatz et al. (1991) Mol. Gen. Genet. 227: 229-237, and U.S. Pat. Nos. 5,814,618 and 5,789,156, herein incorporated by reference.

Where enhanced expression in particular tissues is desired, tissue-specific promoters can be utilized. Tissue-specific promoters include those described by Yamamoto et al. (1997) Plant J. 12(2): 255-265; Kawamata et al. (1997) Plant Cell Physiol. 38(7): 792-803; Hansen et al. (1997) Mol. Gen. Genet. 254(3): 337-343; Russell et al. (1997) Transgenic Res. 6(2): 157-168; Rinehart et al. (1996) Plant Physiol. 112(3): 1331-1341; Van Camp et al. (1996) Plant Physiol. 112(2): 525-535; Canevascni et al. (1996) Plant Physiol. 112(2): 513-524; Yamamoto et al. (1994) Plant Cell Physiol. 35(5): 773-778; Lam (1994) Results Probl. Cell Differ. 20: 181-196; Orozco et al. (1993) Plant Mol. Biol. 23(6): 1129-1138; Mutsuoka et al. (1993) Proc. Natl. Acad. Sci. USA 90 (20): 9586-9590; and Guevara-Garcia et al (1993) Plant J. 4(3): 495-50.

“Operably linked” means joined as part of the same nucleic acid molecule, suitably positioned and oriented for transcription to be initiated from the promoter. DNA operably linked to a promoter is “under transcriptional initiation regulation” of the promoter. In a preferred aspect, the promoter is an inducible promoter or a developmentally regulated promoter.

Particular of interest in the present context are nucleic acid constructs which operate as plant vectors. Specific procedures and vectors previously used with wide success upon plants are described by Guerineau and Mullineaux (1993; Plant transformation and expression vectors. In: Plant Molecular Biology Labfax (Croy RRD ed) Oxford, BIOS Scientific Publishers, pp 121-148). Suitable vectors may include plant viral-derived vectors (see e.g. EP-A-194809).

If desired, selectable genetic markers may be included in the construct, such as those that confer selectable phenotypes such as resistance to antibodies or herbicides (e.g. kanamycin, hygromycin, phosphinotricin, chlorsulfuron, methotrexate, gentamycin, spectinomycin, imidazolinones and glyphosate).

Preferably said promoter is the cinnamate-4-hydroxylase (CH4) promoter, wherein C4H is an enzyme in the phenylpropanoid pathway. Alternatively said promoter is the constitutive promoter, CaMV 35S promoter.

In a further preferred embodiment of the invention the expression of said nucleic acid molecule is down-regulated to reduce glucosyltransferase activity in said cell.

In a preferred embodiment of the invention said expression is reduced by at least 10%. Preferably said activity is reduced by between 10%-99%. Preferably said activity is reduced by at least 20%, 30%, 40%, 50%, 60%, 70%, 80%, or at least 90% when compared to a non-transgenic reference cell.

Preferably said down-regulation is as a result of said cell being null for a nucleic acid molecule selected from the group consisting of;
i) a nucleic acid molecule comprising a nucleic acid sequence as represented in FIG. 1 (SEQ ID NO: 1);
ii) a nucleic acid molecule comprising a nucleic acid sequence which hybridises to the sequence in (i) above;
iii) a nucleic acid molecule comprising a nucleic acid sequences which are degenerate as a result of the genetic code to the sequences defined in (i) and (ii) above.

In using anti-sense genes or partial gene sequences to down-regulate gene expression, a nucleotide sequence is placed under the control of a promoter in a “reverse orientation” such that transcription yields RNA which is complementary to normal mRNA transcribed from the “sense” strand of the target gene. See, for example, Rothstein et al, 1987; Smith et al, (1998), Nature 334, 724-726; Zhang et al (1992) The Plant Cell 4, 1575-1588, English et al. (1996) The Plant Cell 8, 179 188. Antisense technology is also reviewed in Bourque (1995), Plant Science 105, 125-149, and Flavell (1994) PNAS USA 91, 3490-3496.

Preferably said down-regulation is as a result of said cell being null for a nucleic acid molecule selected from the group consisting of;
i) a nucleic acid molecule comprising a nucleic acid sequence as represented in FIG. 1 (SEQ ID NO: 1) and FIG. 3 (SEQ ID NO: 3) and/or FIG. 5 (SEQ ID NO: 5);
ii) a nucleic acid molecule comprising a nucleic acid sequence which hybridises to the sequence in (i) above;
iii) a nucleic acid molecule comprising a nucleic acid sequences which are degenerate as a result of the genetic code to the sequences defined in (i) and (ii) above.

In an alternative embodiment of the invention said down-regulation is the result of said cell being null for a nucleic acid molecule comprising a nucleic acid sequence as shown in FIG. 3 (SEQ ID NO: 3) and FIG. 5 (SEQ ID NO: 5) or a nucleic acid molecule which hybridises to a nucleic acid molecule comprising a nucleic acid sequence as shown in FIG. 3 (SEQ ID NO: 3) and FIG. 5 (SEQ ID NO: 5).

In an alternative preferred embodiment of the invention said cell is transformed with a nucleic acid molecule comprising an expression cassette which cassette comprises a nucleic acid sequence selected from the group consisting of:
i) a nucleic acid molecule comprising a nucleic acid sequence as represented in FIG. 1 (SEQ ID NO: 1);
ii) a nucleic acid molecule comprising a nucleic acid sequence which hybridises to the sequence in (i) above and which glucosylates at least one monolignol;
iii) a nucleic acid molecule comprising a nucleic acid sequences which are degenerate as a result of the genetic code to the sequences defined in (i) and (ii) above,
   wherein said cassette is adapted such that both sense and antisense nucleic acid molecules are transcribed from said cassette.

In a further preferred embodiment of the invention said cassette is provided with at least two promoters adapted to transcribe sense and antisense strands of said nucleic acid molecule.

In a further preferred embodiment of the invention said cassette comprises a nucleic acid molecule wherein said molecule comprises a first part linked to a second part wherein said first and second parts are complementary over at least part of their sequence and further wherein transcription of said nucleic acid molecule produces an RNA molecule which forms a double stranded region by complementary base pairing of said first and second parts.

In a preferred embodiment of the invention said first and second parts are linked by at least one nucleotide base. In a further preferred embodiment of the invention said first and second parts are linked by 2, 3, 4, 5, 6, 7, 8, 9, or 10 nucleotide bases. In a yet further preferred embodiment of the invention said linker is at least 10 nucleotide bases.

In a further preferred embodiment of the invention the length of the RNA molecule or antisense RNA is between 10 nucleotide bases (nb) and 1000 nb. Preferably said RNA molecule or antisense RNA is 100 nb; 200 nb; 300 nb; 400 nb; 500 nb; 600 nb; 700 nb; 800 nb; 900 nb; or 1000 nb in length. More preferably still, said RNA molecule or antisense RNA is at least 1000 nb in length.

More preferably still the length of the RNA molecule or antisense RNA is at least 10 nb; 20 nb; 30 nb; 40 nb; 50 nb; 60 nb; 70 nb; 80 nb; or 90 nb in length. Preferably still said RNA molecule is 21 nb in length.

In an alternative preferred embodiment of the invention said cell is transformed with a nucleic acid molecule comprising an expression cassette(s) which cassette(s) comprises a nucleic acid sequence selected from the group consisting of:
i) a nucleic acid molecule comprising a nucleic acid sequence as represented in FIG. 1 (SEQ ID NO: 1) and FIG. 3 (SEQ ID NO: 3) and/or FIG. 5 (SEQ ID NO: 5);
iii) a nucleic acid molecule comprising a nucleic acid sequence which hybridises to the sequence in (i) above and which glucosylates at least one monolignol;
iii) a nucleic acid molecule comprising a nucleic acid sequences which are degenerate as a result of the genetic code to the sequences defined in (i) and (ii) above,
   wherein said cassette is adapted such that both sense and antisense nucleic acid molecules are transcribed from said cassette.

In a preferred embodiment of the invention said cassette(s) comprises a nucleic molecule comprising a nucleic acid sequence as shown in FIG. 3 (SEQ ID NO: 3) and FIG. 5 (SEQ ID NO: 5) or a nucleic acid molecule which hybridises to a nucleic acid molecule comprising a nucleic acid sequence as shown in FIG. 3 (SEQ ID NO: 3) and FIG. 5 (SEQ ID NO: 5).

In a further preferred embodiment of the invention said expression cassette is part of a vector.

In a preferred embodiment of the invention said transgenic cell is a eukaryotic cell. Preferably said eukaryotic cell is a plant cell. Alternatively said eukaryotic cell is a yeast cell.

In an alternative embodiment of the invention said transgenic cell is a prokaryotic cell. Preferably said prokaryotic cell is a bacterial cell.

In a further preferred embodiment of the invention, a transgenic plant is provided comprising a transgenic cell of the invention.

In yet still a further preferred embodiment of the invention said plant is a woody plant selected from: poplar; eucalyptus; Douglas fir; pine; walnut; ash; birch; oak; teak; and spruce. Preferably said woody plant is a plant used typically in the paper industry, for example poplar.

Methods to transform woody species of plant are well known in the art. For example the transformation of poplar is disclosed in U.S. Pat. No. 4,795,855 and WO9118094. The transformation of eucalyptus is disclosed in EP1050209 and WO9725434. Each of these patents is incorporated in their entirety by reference.

In a still further preferred embodiment of the invention said plant is a non-woody plant selected from the group consisting of: corn (Zea mays), canola (Brassica napus, Brassica rapa ssp.), alfalfa (Medicago sativa), rice (Oryza sativa), rye (Secale cerale), sorghum (Sorghum bicolor, Sorghum vulgare), sunflower (helianthus annuas), wheat (Tritium aestivum), soybean (Glycine max), tobacco (Nicotiana tabacum), potato (Solanum tuberosum), peanuts (Arachis hypogaea), cotton (Gossypium hirsutum), sweet potato (Iopmoea batatus), cassaya (Manihot esculenta), coffee (Cofea spp.), coconut (Cocos nucifera), pineapple (Anana comosus), citris tree (Citrus spp.), cocoa (Theobroma cacao), tea (Camellia senensis), banana (Musa spp.), avocado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), oats, barley, vegetables and ornamentals.

Preferably, plants of the present invention are crop plants (for example, cereals and pulses, maize, wheat, potatoes, tapioca, rice, sorghum, millet, cassaya, barley, pea, and other root, tuber or seed crops) Important seed crops are oil-seed rape, sugar beet, maize, sunflower, soybean, and sorghum. Horticultural plants to which the present invention may be applied include lettuce, endive, vegetable brassicas including cabbage, broccoli and cauliflower, carnations and geraniums. The present invention may be applied in tobacco, cucurbits, carrot, strawberry, sunflower, tomato, pepper and chrysanthemum.

Grain plants that provide seeds of interest include oil-seed plants and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava been, lentils, chickpea, etc.

According to a further aspect of the invention there is provided for modulating the lignin content of a plant comprising the steps of;
i) providing a cell according to the invention,
ii) providing conditions conducive to growth of said cell into a plantlet, and optionally
iii) determining the lignin content of said plant.

According to a fourth aspect of the invention there is provided a method of manufacture of paper or board from a transgenic plant exhibiting an altered lignin content comprising the steps of;
i) pulping the transgenic wood material derived from the transgenic plant according to the invention; and
ii) producing paper from said pulped transgenic wood material.

### BRIEF DESCRIPTION OF THE DRAWINGS

An embodiment of the invention will now be described by example only and with reference to the following figures:

FIG. 1 is the nucleic acid sequence of glucosyltransferase 72E1 (SEQ ID NO: 1);

FIG. 2 is the amino acid sequence of glucosyltransferase 72E1 (SEQ ID NO: 2);

FIG. 3 is the nucleic acid sequence of glucosyltransferase 72E2 (SEQ ID NO: 3);

FIG. 4 is the amino acid sequence of glucosyltransferase 72E2 (SEQ ID NO: 4);

FIG. 5 is the nucleic acid sequence of glucosyltransferase 72E3 (SEQ ID NO: 5)

FIG. 6 is the amino acid sequence of glucosyltransferase 72E3 (SEQ ID NO: 6);

FIG. 7 illustrates examples of monolignols and their modification by the glucosyltransferase 72E1 and 72E2; and

FIG. 8a—A 248 by UTG72E1 cDNA fragment (SEQ ID NO: 13) was amplified using oligos 72E1-5(XhoI/XmaI) (CTCGAGCCCGGGATGAAGATTACAAAAC; SEQ ID NO: 7) and 72E1-3(5-E3) (ATCTTGTCACCACAAAGGCTGATGGGTCG; SEQ ID NO: 8);

FIG. 8b—A 234 by UTG72E3 cDNA fragment (SEQ ID NO: 14) was amplified using oligos 72E3-5(3-E1) (CGACCCATCAGCCTTTGTGGTGACCAAGAT; SEQ ID NO: 9) and 72E3-3(5-E2) (GGTATAGCGAGTGGGTTTCGTTGCACTGTG; SEQ ID NO: 10);

FIG. 8c—A 247 by UTG72E2 cDNA fragment (SEQ ID NO: 15) was amplified using oligos 72E2-5(3-E3) (CACAGTGCAACGAAACCCACTCGCTATACC; SEQ ID NO: 11) and 72E2-3(XbaI/SwaI) (ATTTAAATTCTAGAGATGATTGTATCGGTCTG; SEQ ID NO: 12) nucleic acid sequences are presented in FIGS. 8a-8c.

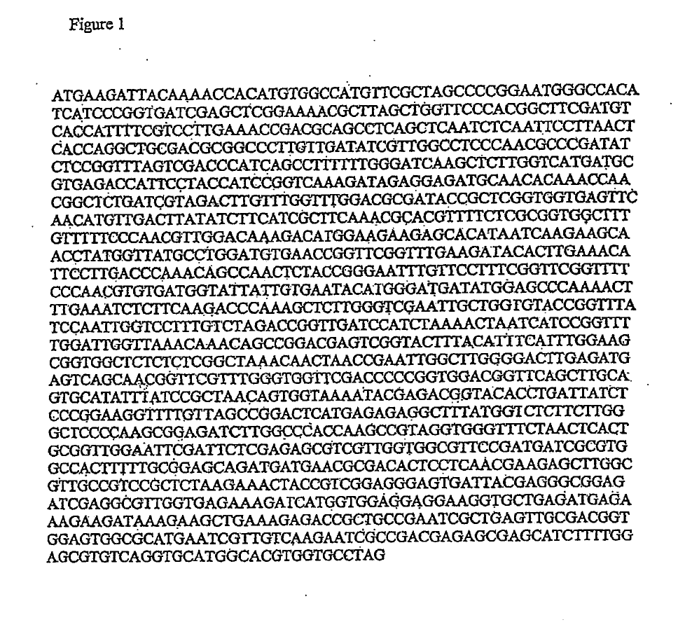

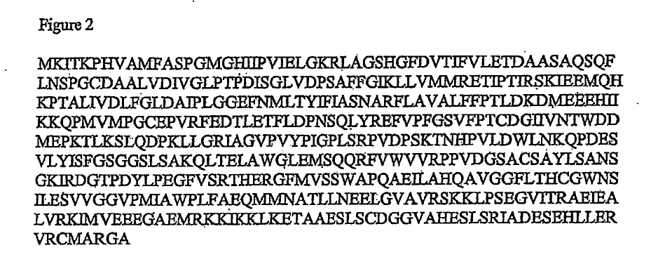

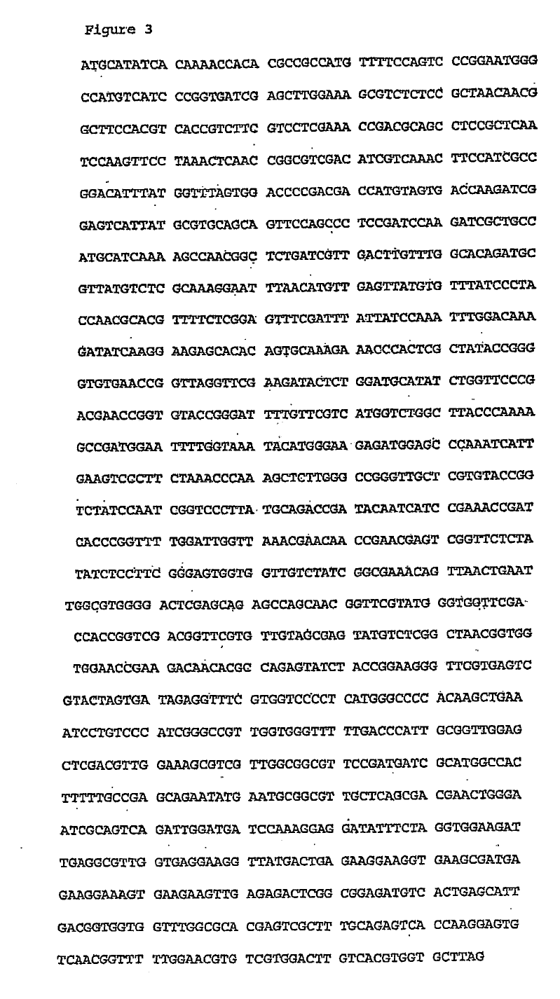

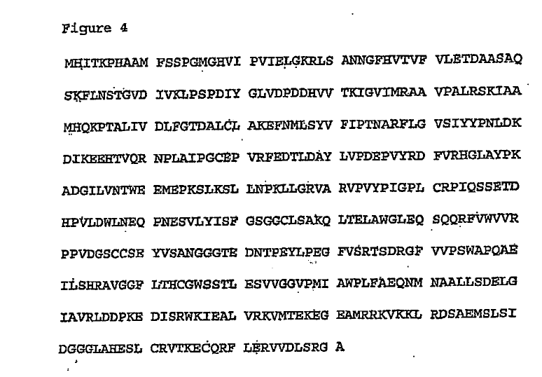

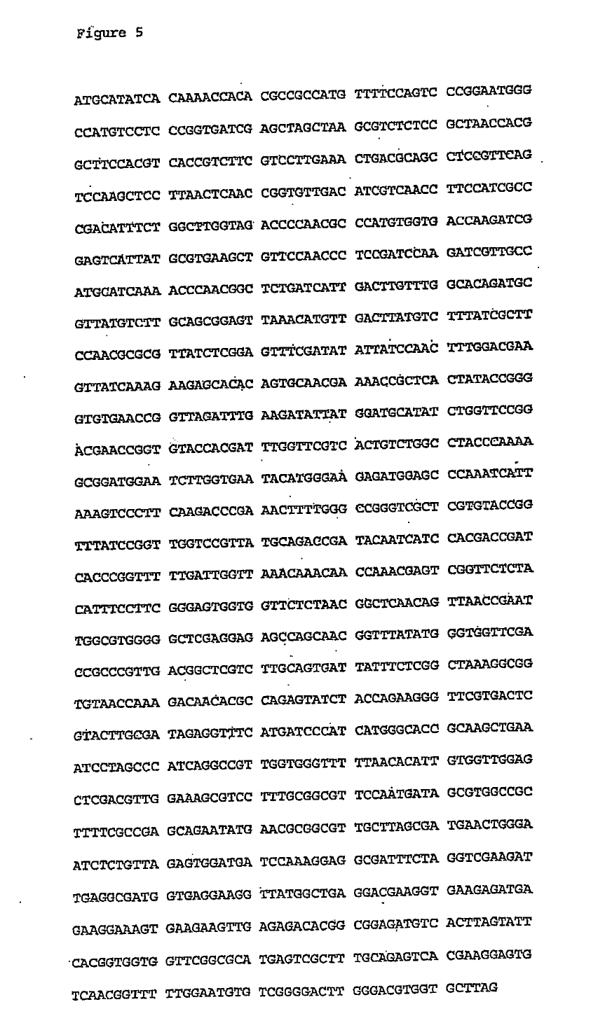

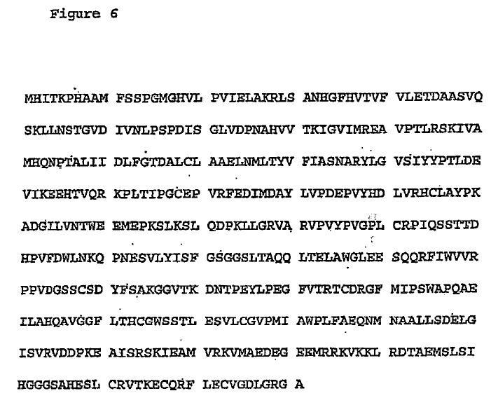

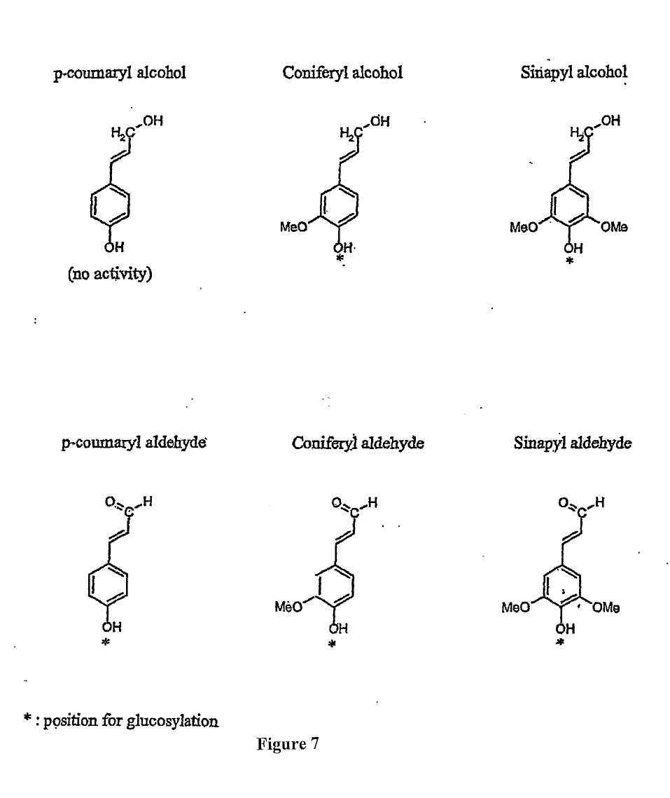

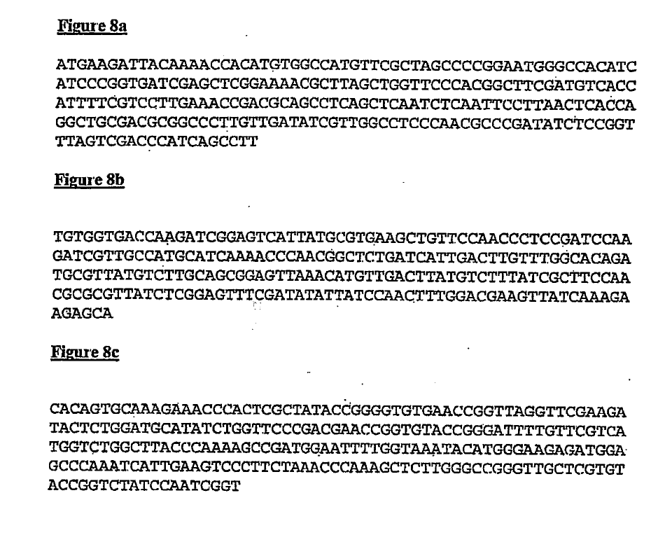

### DETAILED DESCRIPTION

### Glucosyltransferase Activity Assay

Recombinant UGT72E1 was expressed and purified from E. coli as described previously (Lim et al., 2001, J. Biol. Chem. 276, 4344-4349). The enzyme (2 μg) was incubated with 1 mM phenolic substrates, 5 mM UDP-glucose, 100 mM Tris-HCl, pH 7.0 in a total volume 200 μl. The reaction mix was incubated at 30° C. for 1 h and was analysed using HPLC subsequently.

### HPLC Analysis

Coniferyl alcohol: 10-25% acetonitrile (0.1% TFA), 306 nm
Sinapyl alcohol: 10-25% acetonitrile (0.1% TFA), 285 nm
p-coumaryl alcohol: 10-25% acetonitrile (0.1% TFA), 311 nm
coniferyl aldehyde: 10-47% acetonitrile (0.1% TFA), 311 nm
sinapyl aldehyde: 10-47% acetonitrile (0.1% TFA), 280 nm
p-coumaryl aldehyde: 10-47% acetonitrile (0.1% TFA), 315 nm

Table 1 illustrates the activity of 72E1 with respect to monolignol substrates.

Activity (area, uv × sec)

| Substrates | 72E1 | 72E2 |
|---|---|---|
| Coniferyl alcohol | 3225766 | 29756923 |
| Sinapyl alcohol | 0 | 3339410 |
| p-coumaryl alcohol | 0 | 0 |
| Coniferyl aldehyde | 21950129 | 5068215 |
| Sinapyl aldehyde | 13655427 | 37002362 |
| p-coumaryl aldehyde | 9243651 | 2612331 |

### RNA Silencing of UGT72E1, UGT72E2 and UGT72E3

The UGT72E1 and UGT72E3 fragments were linked by standard procedure taking advantage of the overlapping sequences of oligos 72E1-3(5-E3) and 72E3-5(3-E1) and further PCR amplification using oligos 72E1-5(XhoI/XmaI) and 72E3-3(5-E2). Then the UGT72E1E3 fragment were linked to the UGT72E2 fragment by, again, taking advantage of the overlapping sequence of oligos 72E3-3(5-E2) and 72E2-5(3-E3) and further PCR amplification with oligos 72E1-5(XhoI/XmaI) and 72E2-3(XbaI/SwaI).

The UGT72E1E3E2 fragment was then cloned into the pGEM-T vector (Promega). From that vector, the fragment was excised by XbaI/XmaI double digestion and cloned into pFGC5941 open with the same restriction enzymes. Then the fragment UGT72E1E3E2 was excised from the pGEM-T construct with a XhoI/SwaI double digestion and cloned into XhoI/SwaI-digested pFGC5941 vector (carrying the previously cloned XbaI/XmaI UGT72E1E3E2 fragment).

The resulting 72E132 inverted repeat construct was used to transform A. thaliana (Columbia ecotype) plants using standard floral-dipping methods.

Kanamycin resistant plants were selected in media containing the antibiotic. Some (10 out of 40) of the T1 primary transformants showed more elongated petioles and a smaller plant size compared to non-transformed plants. We are currently selecting for T3 homozygous plants. These plants will be assessed for RNA levels of the targeted UGT mRNAs and then a more deep analysis of secondary metabolite population will be conducted.

1. A transgenic cell wherein the genome of said cell comprises a nucleic acid molecule selected from the group consisting of:
i) a nucleic acid molecule comprising a nucleic acid sequence as represented in SEQ ID NO: 1;
ii) a nucleic acid molecule comprising a nucleic acid sequence which hybridizes under stringent conditions to the sequence in (i) above and which glucosylates at least one monolignol; and
iii) a nucleic acid molecule comprising a nucleic acid sequence which is degenerate as a result of the genetic code to the sequences defined in (i) and (ii) above.

2. The cell according to claim 1 wherein said monolignol is selected from the group consisting of; p-coumaryl aldehyde, coniferyl aldehyde, and sinapyl aldehyde.

3. The cell according to claim 1, wherein said nucleic acid molecule comprises a nucleic acid sequence as shown in SEQ ID NO: 1.

4. The cell according to claim 1, wherein said nucleic acid molecule consists of a nucleic acid sequence as shown in SEQ ID NO: 1.

5. The cell according to claim 1 wherein said nucleic acid molecule is over expressed.

6. The cell according to claim 5 wherein said cell over-expresses a nucleic acid molecule as represented by the nucleic acid sequence shown in—SEQ ID NO: 3 and SEQ ID NO: 5, or a nucleic acid molecule which hybridises to a nucleic acid molecule as represented by the nucleic acid sequence in SEQ ID NO: 3 and SEQ ID NO: 5.

7. The cell according to claim 1 wherein the expression of said nucleic acid molecule is down-regulated to reduce glucosyltransferase activity in said cell.

8. The cell according to claim 7 wherein said down-regulation is as a result of said cell being null for a nucleic acid molecule selected from the group consisting of;
i) a nucleic acid molecule comprising a nucleic acid sequence as represented in SEQ ID NO: 1;
ii) a nucleic acid molecule comprising a nucleic acid sequence which hybridises to the sequence in (i) above; and
iii) a nucleic acid molecule comprising a nucleic acid sequences which are degenerate as a result of the genetic code to the sequences defined in (i) and (ii) above.

9. The cell according to claim 7, wherein said down-regulation is as a result of said cell being null for a nucleic acid molecule selected from the group consisting of;
i) a nucleic acid molecule comprising a nucleic acid sequence as represented in SEQ ID NO: 1 and SEQ ID NO: 3 and/or SEQ ID NO: 5;
ii) a nucleic acid molecule comprising a nucleic acid sequence which hybridises to the sequence in (i) above; and
iii) a nucleic acid molecule comprising a nucleic acid sequences which are degenerate as a result of the genetic code to the sequences defined in (i) and (ii) above.

10. The cell according to claim 7, wherein said down-regulation is the result of said cell being null for a nucleic acid molecule comprising a nucleic acid sequence as shown in SEQ ID NO: 3 and SEQ ID NO: 5, or a nucleic acid molecule which hybridizes to a nucleic acid molecule comprising a nucleic acid sequence as shown in SEQ ID NO: 3 and SEQ ID NO: 5.

11. The cell according to claim 7, wherein said cell is transformed with a nucleic acid molecule comprising an expression cassette which cassette comprises a nucleic acid sequence selected from the group consisting of:
i) a nucleic acid molecule comprising a nucleic acid sequence as represented in SEQ ID NO: 1;
ii) a nucleic acid molecule comprising a nucleic acid sequence which hybridises to the sequence in (i) above and which glucosylates at least one monolignol; and
iii) a nucleic acid molecule comprising a nucleic acid sequences which are degenerate as a result of the genetic code to the sequences defined in (i) and (ii) above,
wherein said cassette is adapted such that both sense and antisense nucleic acid molecules are transcribed from said cassette.

12. The cell according to claim 11 wherein said cassette is provided with at least two promoters adapted to transcribe sense and antisense strands of said nucleic acid molecule.

13. The cell according to claim 11 wherein said cassette comprises a nucleic acid molecule wherein said molecule comprises a first part linked to a second part wherein said first and second parts are complementary over at least part of their sequence and further wherein transcription of said nucleic acid molecule produces an RNA molecule which forms a double stranded region by complementary base pairing of said first and second parts.

14. The cell according to claim 13 wherein said first and second parts are linked by at least one nucleotide base.

15. The cell according to claim 7 wherein said cell is transformed with a nucleic acid molecule comprising an expression cassette which cassette comprises a nucleic acid sequence selected from the group consisting of:
i) a nucleic acid molecule comprising a nucleic acid sequence as represented in SEQ ID NO: 1 and SEQ ID NO: 3 and/or SEQ ID NO: 5;
ii) a nucleic acid molecule comprising a nucleic acid sequence which hybridises to the sequence in (i) above and which glucosylates at least one monolignol; and
iii) a nucleic acid molecule comprising a nucleic acid sequences which are degenerate as a result of the genetic code to the sequences defined in (i) and (ii) above,
wherein said cassette is adapted such that both sense and antisense nucleic acid molecules are transcribed from said cassette.

16. The cell according to claim 15 wherein said cassette comprises a nucleic molecule comprising a nucleic acid sequence as shown in SEQ ID NO: 3 and SEQ ID NO: 5 or a nucleic acid molecule which hybridises to a nucleic acid molecule comprising a nucleic acid sequence as shown in SEQ ID NO: 3 and SEQ ID NO: 5.

17. The cell according to claim 1 wherein said transgenic cell is a eukaryotic cell.

18. The cell according to claim 17 wherein said cell is a plant cell.

19. A transgenic plant comprising the cell according to claim 18.

20. The plant according to claim 19 wherein said plant is a woody plant selected from: poplar; eucalyptus; Douglas fir; pine; walnut; ash; birch; oak; teak; and spruce.

21. A method for modulating the lignin content of a plant comprising;
i) providing the cell of claim 1,
ii) providing conditions conducive to growth of said cell into a plantlet, and
iii) determining the lignin content of said plant.

22. A method of manufacture of paper or board from a transgenic plant exhibiting an altered lignin content comprising;
i) pulping the transgenic wood material derived from the transgenic plant according to claim 19; and
ii) producing paper from said pulped transgenic wood material.
